(19) United States
(12) Patent Application Publication
(10) Pub. No.: US 2002/0091338 A1
(43) Pub. Date: Jul. 11, 2002

(54) Medical guidewire and method for making

(76) Inventor: Jack R. Frautschi, Athens, TX (US)

Correspondence Address: Bush Intellectual Property Law Group, LLC, P.O. Box 381146, Birmingham, AL, 35242, US

(21) Appl. No.: 10/005,098

(22) Filed: Dec. 5, 2001

Related U.S. Application Data
(60) Provisional application No. 60/251,489, filed on Dec. 5, 2000.
(60) Provisional application No. 60/324,234, filed on Sep. 21, 2001.

(51) Int. Cl. A61B 5/00

(52) U.S. Cl. 600/585

(57) ABSTRACT

The present invention includes a medical guidewire having improved torque transfer from the proximal end of the guidewire to its distal end. The guidewire comprises a radiopaque coil affixed about its distal end and a flexible polymer cladding over the distal end of the wire, wherein the coil comprises a plurality of spirals having gaps therebetween such that the cladding fills the gaps between the spirals and adheres to the wire. The flexible cladding is colored to assist operators in quickly differentiating the distal end of the wire from its proximal end and the color of the cladding is selected from a predetermined color scheme to indicate the particular size of the guidewire. A method for forming a curved distal tip comprises the steps of urging a blade against a first surface of the wire, urging an opposing force against an opposing second surface of the wire immediately opposite the blade, and pulling the wire over the blade such that the first surface is stressed by the blade, thereby causing the wire to curve towards the stressed area.

### REFERENCE TO OTHER PATENT APPLICATIONS

This non-provisional patent application claims priority to U.S. Provisional Patent Application No. 60/251,489, filed Dec. 5, 2000, and U.S. Provisional Patent Application No. 60/324,234, filed Sep. 21, 2001. The entire texts of both applications are incorporated herein by reference.

### FIELD OF THE INVENTION

The present invention is directed to a medical guidewire and a method for shaping a medical guidewire. More particularly, the present invention is directed to a medical guidewire having improved torque transfer from the proximal end of the guidewire to its distal end, a color-coding system for medical guidewires to assist operators in quickly differentiating the proximal end of a guidewire from its distal end and determining the size of the guidewire, and a method for forming a curved distal tip on a medical guidewire.

### BACKGROUND OF THE INVENTION

Medical guidewires are ubiquitously used by physicians and medical technicians (“operators”) to assist with the insertion of catheters and other devices into patients during medical procedures. These guidewires typically comprise a flexible “pseudoelastic” wire made from a shape memory alloy, such as Nitinol (nickel-titanium alloy), to avoid kinking during insertion. Further, the distal ends of prior art guidewires are commonly curved (e.g. a “J-tip”) to avoid injury to the vasculature as well as to facilitate navigation therethrough.

As an operator navigates a guidewire through a patient's vasculature, the operator can rotate the proximal end of the guidewire, which, in turn, rotates the distal end of the guidewire to allow the guidewire to be navigated through the tortuous vasculature. It is preferable that rotation at the proximal end of the guidewire translates perfectly to its distal end, resulting in 1:1 torque transfer.

To place a curve in the distal end of a pseudoelastic wire, present methods include shaping the wire to the desired shape and annealing the wire with heat to set the shape. A problem encountered with using heat to anneal a pseudoelastic wire is that the transition temperature (A_{f}) at which the wire becomes pseudoelastic increases. To retain the curved shape at body temperature (37° C.) by annealing the wire, the transition temperature must be raised above 37° C. As a result of having the transition temperature above 37° C., the curve no longer exhibits pseudoelastic properties at body temperature. Consequently, the end of a curved guidewire shaped by this method will be less flexible than desired.

To assist the operator in navigating a guidewire, the distal end of the wire commonly comprises a radiopaque spiral coil affixed thereabout. The radiopaque coil allows the tip of the guidewire to be visualized radiographically during medical procedures. The coil is typically affixed only at its ends to the wire, so that the coil will not compromise the flexibility of the distal end of the wire. Because the coil is not affixed to the wire along the coil's entire length, the coil has a tendency to slightly wind or unwind as the wire is rotated. As a result, the torque transfer from the proximal end of the guidewire to its distal end is dampened (i.e., less than 1:1 torque transfer).

Prior art guidewires also commonly comprise a polymer cladding over the wire and a lubricious polymer coating over the cladding to reduce the risk of injury to the vasculature as the guidewire is advanced therethrough. To achieve 1:1 torque transfer with a polymer cladded guidewire, it is necessary that the polymer cladding be adequately adhered to the core wire, so that the core wire and polymer cladding move as a single unit. In prior art polymer cladded guidewires having radiopaque coils, the polymer cladding is applied over the exterior of the coil and is prevented from contacting the core wire passing through the coil. This is because the “unextended” coil is wound in such a manner that the adjacent spirals are in abutment. As a result, the torque transfer from the proximal end of the guidewire to its distal end is further dampened.

Prior art guidewires are available in a number of different sizes, including both lengths and diameters. During medical procedures, it is common to have a plurality of different guidewires set out in a guidewire bowl for use. This can prove frustrating to the operator when trying to quickly determine the proximal end of the guidewire from its distal end and the size of the guidewire.

Accordingly, there is a need for medical guidewires and methods for making guidewires which overcome these problems found with prior art medical guidewires.

### SUMMARY OF THE INVENTION

The present invention includes a medical guidewire providing improved torque transfer from the proximal end of the guidewire to its distal end. The guidewire comprises a radiopaque coil affixed about its distal end and a flexible polymer cladding over the distal end of the wire, wherein the coil comprises a plurality of spirals having gaps therebetween such that the cladding fills the gaps between the spirals and adheres to the core wire. As a result, the torque transfer from the proximal end of the guidewire to its distal end approaches 1:1 torque transfer without sacrificing flexibility.

The flexible cladding is preferably colored to assist operators in quickly differentiating the distal end of the wire from its proximal end and the color of the cladding is selected from a predetermined color scheme to indicate the particular size of the guidewire.

A method for forming a curved distal tip comprises the steps of urging a blade against a first surface of the wire, urging an opposing force against an opposing second surface of the wire immediately opposite the blade, and pulling the wire over the blade such that the first surface is stressed by the blade, thereby causing the wire to curve towards the stressed area. As the force from the blade is increased, the radius of curvature of the curve decreases. The process is analogous to the practice of running a blade from a pair of scissors across ribbon to coil the ribbon. This “cold forming” technique allows shaping of the wire without using heat or changing its original transitional temperature (A_{f}). The transition temperature of pseudoelastic material is normally below body temperature so the alloy is pseudoelastic at its use temperature. As a consequence, the tip of the wire retains its flexibility/pseudoelasticity at body temperature.

Other features and advantages of the present invention will become apparent from the following detailed description and the appended drawings.

### BRIEF DESCRIPTION OF DRAWINGS

A medical guidewire incorporating features of the present invention is shown in the attached drawings which form a portion of the disclosure and wherein:

FIG. 1 is an elevated perspective view of the distal end of a prior art guidewire having an unextended spiral coil.

FIG. 2 is an elevated perspective view of the distal end of a guidewire according to the present invention having an extended spiral coil.

FIGS. 3A-3C are schematic representations of a blade engaging the distal end of a guidewire to form a curved end.

### DESCRIPTION OF PREFERRED EMBODIMENTS

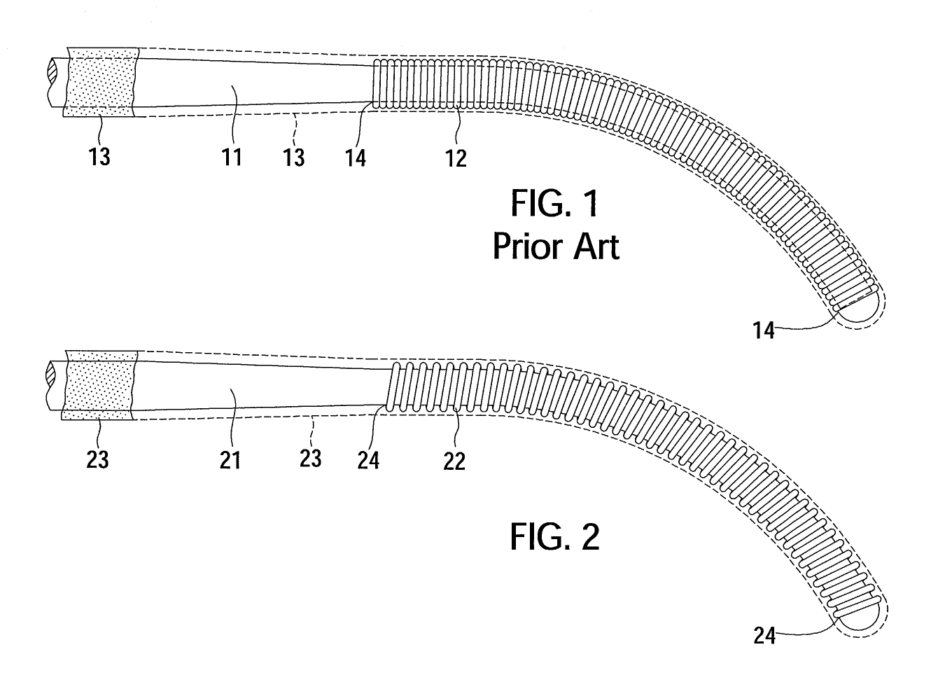

Referring to the drawings for a better understanding of the invention, FIG. 1 illustrates a prior art guidewire comprising a core wire 11 having a spiral coil 12 affixed about its distal end and a polymer cladding 13 over the wire 11 and coil 12. The coil 12 is welded to the wire 11 at points 14. The coil is in an “unextended” configuration such that the adjacent spirals are in abutment and the polymer cladding does not contact the core wire encompassed by the coil.

FIG. 2 illustrates one embodiment of the guidewire of the present invention, wherein the guidewire preferably comprises a core wire 21 having a spiral coil 22 affixed about its distal end wherein the coil is in an “extended” configuration such that the adjacent spirals are not in abutment. The wire 21 preferably comprises a pseudoelastic material, such as a nickel-titanium (NiTi) alloy or a copper-based alloy such as copper-zinc-aluminum (CuZnAl) or copper-aluminum-nickel (CuAlNi). The coil 22 is preferably welded to the wire 21 at points 24 and preferably comprises a radiopaque material, such as platinum or tungsten or a combination thereof. The guidewire preferably further comprises a flexible polymer cladding 23 over the wire 21 and coil 22 that permeates the spaces between the spirals of the coil to adhere to the wire 21, thereby encapsulating the coil 22. As a result, the torque transfer from the proximal end of the guidewire to its distal end approaches 1:1 torque transfer without sacrificing flexibility. Flexible polymer cladding suitable for the present invention are well known in the art.

The guidewires according to the present invention preferably include a colorant incorporated within the polymer cladding 23 at the distal end of the guidewire, thus allowing a quick visual determination of both the size (length and/or diameter) and orientation of the guidewire. The particular color of the cladding is selected from a predetermined color scheme to indicate the particular size of the guidewire.

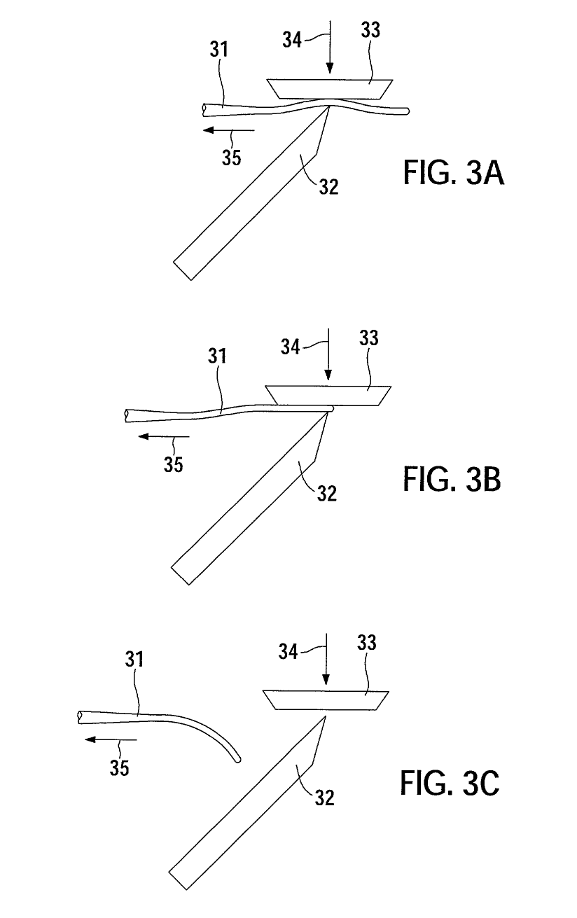

In the method of forming the curved guidewire tip of the present invention, as illustrated in FIGS. 3A-3C, no heat is involved and the distal end is not annealed. Rather, a knife-like blade 32 is urged against one surface of the wire 31, preferably against the proximal portion of the area of the wire to be curved. The blade 32 is preferably angled approximately 45° against the surface of the wire 31. The angle of the blade may also be changed between 5-90° to change the radius of the curve. A predetermined force, preferably 1-15 lbs of perpendicular force, is applied from the blade 32 against the wire 31. An opposing force 34 in the form of, for example, a silicon padded plate 33, is urged against a second surface of the wire 31 immediately opposite the blade 32.

The wire is slid in direction 35 across the blade 32. Alternatively, the blade 32 can be slid distally along the wire 31 along the area to be curved. The movement of the blade 32 along one side of the wire 31 places a surface stress on that side of the wire, causing the wire to bend or curve towards the side where the stress has been applied. As the force on the blade is increased the radius of curvature of the curve decreases. The process is analogous to the practice of running a blade from a pair of scissors across ribbon to coil the ribbon.

This “cold forming” technique allows shaping of the wire without changing its original transitional temperature (A_{f}). Thus, the wire shape remains pseudoelastic and its original flexibility is retained. The cold forming technique also allows the tip of the wire to be much softer than a tip formed with heat, at the same tip diameter. It would be necessary to decrease the guidewire tip diameter to achieve the same softness when using the heat process to form the tip. However, decreasing the tip diameter any further increases safety concerns for the wire, due to the reduced tensile strength of the tip.

Although the invention has been described in various forms, it should be understood that the invention is not so limited but is susceptible of various changes and modifications without departing from the sprit thereof. For example, it is anticipated that the described method for “cold-forming” a shape into a shape memory alloy would find application in other technologies where there is a need of shaping the alloy without changing its transition temperature (A_{f}) and thereby decreasing its flexibility.

1. A medical guidewire, comprising:
a. an elongated wire having a proximal end and a distal end;
b. an extended spiral coil affixed about said distal end of said wire, wherein said coil comprises a plurality of spirals having gaps therebetween; and
c. a flexible cladding over said distal end of said wire and said coil, wherein said cladding fills said gaps between said spirals and adheres to said wire within said coil.

2. A medical guidewire according to claim 1, wherein said wire comprises a pseudoelastic material.

3. A medical guidewire according to claim 2, wherein said pseudoelastic material is selected from the group of shape memory alloys consisting of NiTi, CuZnAl and CuAlNi.

4. A medical guidewire according to claim 1, wherein said coil is radiopaque.

5. A medical guidewire according to claim 1, wherein said flexible cladding over said distal end of said wire is colored to assist operators in quickly differentiating said distal end of said wire from said proximal end.

6. A medical guidewire according to claim 5, wherein the color of said cladding is selected from a predetermined color scheme to indicate the particular size of said guidewire.

7. A medical guidewire according to claim 6, wherein said color scheme comprises a plurality of colors corresponding to different sizes of medical guidewires.

8. A color-coding system for medical guidewires, comprising a color scheme comprising a plurality of colors corresponding to different sizes of medical guidewires, wherein a color from said color scheme is incorporated into a cladding over a guidewire to indicate the particular size of said guidewire.

9. A method for forming a curved distal tip in a medical guidewire wire, comprising the steps of:
a. urging a blade against a first surface of said wire at a distal end thereof;
b. urging an opposing force against an opposing second surface of said wire immediately opposite said blade; and
c. pulling said wire over said blade such that said first surface is stressed by said blade, thereby causing said wire to curve towards the stressed area.

10. A method according to claim 9, wherein said blade is loaded between 1-15 lbs of force at a 45° angle to said first surface as said wire is pulled over said blade.

11. A method for forming a curved distal tip in a medical guidewire wire, comprising the steps of:
a. urging a blade against a first surface of said wire at a distal end thereof;
b. urging an opposing force against an opposing second surface of said wire immediately opposite said blade; and
c. sliding said blade along said first surface such that said first surface is stressed by said blade, thereby causing said wire to curve towards the stressed area.

12. A method according to claim 11, wherein said blade is loaded between 1-15 lbs of force at a 45° angle to said first surface as said blade is slide along said first surface.

13. A method for forming a curved surface in a shape memory material, comprising the steps of:
a. urging a blade against a first surface of said shape memory material;
b. urging an opposing force against an opposing second surface of said shape memory material immediately opposite said blade; and
c. pulling said shape memory material over said blade such that said first surface is stressed by said blade, thereby causing said shape memory material to curve towards the stressed area.

14. A method according to claim 13, wherein said blade is loaded between 1-15 lbs of force at a 45° angle to said first surface as said shape memory material is pulled over said blade.

15. A method for forming a curved surface in a shape memory material, comprising the steps of:
a. urging a blade against a first surface of said shape memory material;
b. urging an opposing force against an opposing second surface of said shape memory material immediately opposite said blade; and
c. sliding said blade along said first surface such that said first surface is stressed by said blade, thereby causing said shape memory material to curve towards the stressed area.

16. A method according to claim 15, wherein said blade is loaded between 1-15 lbs of force at a 45° angle to said first surface as said blade is slide along said first surface.
